(19) United States
(12) Patent Application Publication
(10) Pub. No.: US 2014/0248252 A1
(43) Pub. Date: Sep. 4, 2014

(54) New Salt And Medical Use

(71) Applicant: Pfizer Limited, Sandwich (GB)

(72) Inventors: Zahid Ali, Great Abington (UK); Kenneth John Butcher, Sandwich, Kent (UK); Richard Philip Butt, Great Abington (UK); Stephen John Felstead, Sandwich, Kent (UK); Sophie Glatt, Great Abington (UK); Ruth Mitchell McKernan, Great Abington (UK); Maninder Panesar, Sandwich, Kent (UK)

(73) Assignee: PFIZER LIMITED, Sandwich (GB)

(21) Appl. No.: 14/351,649

(22) PCT Filed: Oct. 19, 2012
(86) PCT No.: PCT/IB2012/055760

§ 371 Date: Apr. 14, 2014

Related U.S. Application Data
(60) Provisional application No. 61/700,582, filed on Sep. 13, 2012.
(60) Provisional application No. 61/549,823, filed on Oct. 21, 2011.

(51) Int. Cl. C07D 417/12; A61K 31/427; A61K 45/06; C07C 309/30

(52) U.S. Cl. CPC C07D 417/12; C07C 309/30; A61K 31/427; A61K 45/06
USPC 424/94.4; 514/370; 548/197

(57) ABSTRACT

The present invention is directed to 4-[2-(5-amino-1H-pyrazol-4-yl)-4-chlorophenoxy]-5-chloro-2-fluoro-N-(1,3-thiazol-4-yl)benzenesulfonamide or a pharmaceutically acceptable salt thereof for the treatment of a disease associated with elevated blood uric acid levels, such as hyperuricemia or gout. The invention is also directed to the tosylate salt of 4-[2-(5-amino-1H-pyrazol-4-yl)-4-chlorophenoxy]-5-chloro-2-fluoro-N-(1,3-thiazol-4-yl)benzenesulfonamide.

The Invention relates to a new medical use for 4-[2-(5-amino-1H-pyrazol-4-yl)-4-chlorophenoxy]-5-chloro-2-fluoro-N-(1,3-thiazol-4-yl)benzenesulfonamide, to an improved pharmaceutically acceptable salt of 4-[2-(5-amino-1H-pyrazol-4-yl)-4-chlorophenoxy]-5-chloro-2-fluoro-N-(1,3-thiazol-4-yl)benzenesulfonamide and to compositions thereof.

The compound 4-[2-(5-amino-1H-pyrazol-4-yl)-4-chlorophenoxy]-5-chloro-2-fluoro-N-(1,3-thiazol-4-yl)benzenesulfonamide is a voltage-gated sodium channel (Naᵥ) inhibitor, more specifically a Naᵥ 1.7 inhibitor, and is disclosed as Example 788 in international patent application publication number WO2010/079443, the entire contents of which are incorporated herein by reference. As a Naᵥ 1.7 inhibitor the compound is potentially useful in the treatment of a wide range of disorders, particularly pain, including: acute pain; chronic pain; neuropathic pain; inflammatory pain; visceral pain; nociceptive pain including post-surgical pain; and mixed pain types involving the viscera, gastrointestinal tract, cranial structures, musculoskeletal system, spine, urogenital system, cardiovascular system and CNS, including cancer pain, back and orofacial pain.

Uric acid is the final product of purine metabolism in humans. In humans, unlike many other animals, uric acid is not further broken down, but is predominantly (70%) excreted into the urine with the remaining 30% excreted in faeces. Hyperuricemia is defined as an excessive production or decreased excretion of uric acid and can occur as an overproduction or under excretion of serum uric acid (sUA), or a combination of the both. Renal under excretion of uric acid is the primary cause of hyperuricemia in about 90% of cases, while overproduction is the cause in less than 10%. Increased sUA concentration above than 6.8 mg/dL results in crystallisation of uric acid in the form of salts, such as monosodium urate, and to precipitation of these crystals in joints, on tendons and in the surrounding tissues. These crystals (known as tophi) trigger a local immune-mediated inflammatory reaction, leading to gout. The risk of gout increases with increased sUA levels.

Renal under excretion of uric acid is the primary cause of hyperuricemia in about 90% of cases, while overproduction is the cause in less than 10%. The risk of gout increases with increased uric acid levels.

Gout is a painful condition that can present in a number of ways, although the most usual is a recurrent attack of acute inflammatory arthritis (a red, tender, hot, swollen joint) often occurring in big toes, heels, knees, wrists and fingers.

Gout is treated by agents to both decrease the cause and effects of uric acid crystal inflammation and pain.

The pain associated with gout is commonly treated with pain and anti-inflammatory drugs such as nonsteroidal anti-inflammatory drugs (NSAIDs), colchicine and steroids. Agents that decrease sUA levels may be used to treat the cause of gout. These include agents that: inhibit the enzymes that result in uric acid production, such as xanthine oxidase inhibitors (e.g. allopurinol, febuxostat or tisopurine), or purine nucleoside phosphorylase (PNP) inhibitors (e.g. ulodesine); metabolise uric acid, such as urate oxidases—also known as uricases (e.g. pegloticase); or increase the excretion of uric acid in the urine (uricosurics), Uricosurics include agents that inhibit the transporters responsible for renal reabsorption of uric acid back into the blood, such as benziodarone, isobromindione, probenecid and sulphinpyrazone, and URAT-1 inhibitors (e.g. benzbromarone).

URAT-1 is also known as solute carrier family 22 (organic anion/cation transporter), member 12, and is encoded by the gene SLC22A12. Human genetic analysis has demonstrated that polymorphisms in the SLC22A12 gene are directly associated with changes in serum uric acid. Inhibitors of uric acid transport, such as URAT-1, are therefore effective in the treatment of gout.

There is a continuing need to provide new treatments for gout that are more effective and/or are better tolerated.

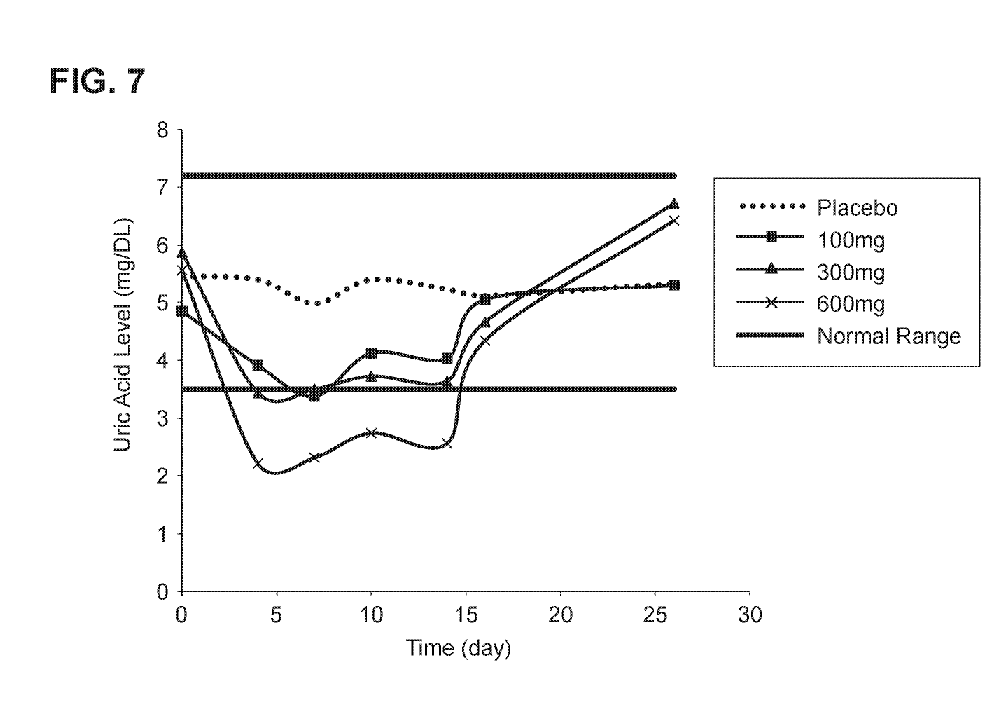

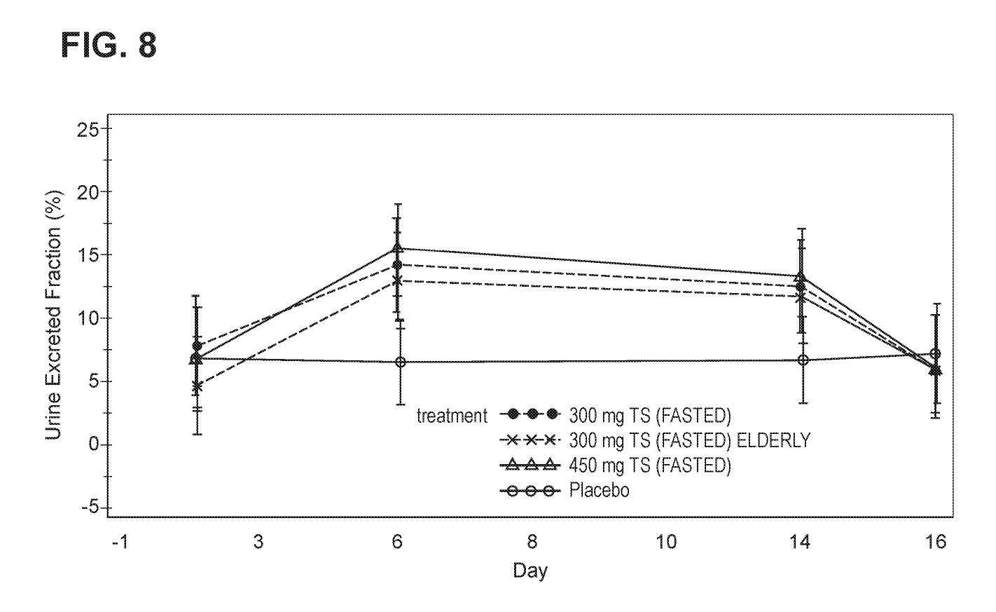

Surprisingly, it has now been found that 4-[2-(5-amino-1H-pyrazol-4-yl)-4-chlorophenoxy]-5-chloro-2-fluoro-N-(1,3-thiazol-4-yl)benzenesulfonamide reduces blood uric acid levels. As shown herein, 4-[2-(5-amino-1H-pyrazol-4-yl)-4-chlorophenoxy]-5-chloro-2-fluoro-N-(1,3-thiazol-4-yl)benzenesulfonamide is an inhibitor of URAT-1. This uric acid lowering effect is discussed in more detail hereinafter with reference to the data in Tables 5 to 9 and FIGS. 7 and 8. These data were obtained using oral dispersions prepared from 4-[2-(5-amino-1H-pyrazol-4-yl)-4-chlorophenoxy]-5-chloro-2-fluoro-N-(1,3-thiazol-4-yl)benzenesulfonamide and the tosylate salt thereof.

4-[2-(5-Amino-1H-pyrazol-4-yl)-4-chlorophenoxy]-5-chloro-2-fluoro-N-(1,3-thiazol-4-yl)benzenesulfonamide is therefore useful in the treatment of diseases associated with high blood uric acid levels such as hyperuricemia, including renal disorders associated with hyperuricemia (e.g. urinary calculi); and gout, including gouty tophus and gouty arthritis. It also follows that 4-[2-(5-amino-1H-pyrazol-4-yl)-4-chlorophenoxy]-5-chloro-2-fluoro-N-(1,3-thiazol-4-yl)benzenesulfonamide is useful in the treatment of a disease where a URAT-1 inhibitor is indicated.

In a first aspect the invention provides 4-[2-(5-amino-1H-pyrazol-4-yl)-4-chlorophenoxy]-5-chloro-2-fluoro-N-(1,3-thiazol-4-yl)benzenesulfonamide or a pharmaceutically acceptable salt thereof for the treatment of a disease associated with elevated blood uric acid levels.

In one embodiment the disease associated with elevated blood uric acid levels is hyperuricemia.

In another embodiment the disease associated with elevated blood uric acid levels is gout.

In another aspect the invention provides 4-[2-(5-amino-1H-pyrazol-4-yl)-4-chlorophenoxy]-5-chloro-2-fluoro-N-(1,3-thiazol-4-yl)benzenesulfonamide or a pharmaceutically acceptable salt thereof for the treatment of a disease where a URAT-1 inhibitor is indicated.

In another aspect the invention provides the use of 4-[2-(5-amino-1H-pyrazol-4-yl)-4-chlorophenoxy]-5-chloro-2-fluoro-N-(1,3-thiazol-4-yl)benzenesulfonamide or a pharmaceutically acceptable salt thereof in the manufacture of a medicament for the treatment of a disease associated with elevated blood uric acid levels.

In another aspect the invention provides the use of 4-[2-(5-amino-1H-pyrazol-4-yl)-4-chlorophenoxy]-5-chloro-2-fluoro-N-(1,3-thiazol-4-yl)benzenesulfonamide or a pharmaceutically acceptable salt thereof in the manufacture of a medicament for the treatment of a disease where a URAT-1 inhibitor is indicated.

In another aspect the invention provides a method of treating a disease associated with elevated blood uric acid levels which comprises administering an effective amount of 4-[2-(5-amino-1H-pyrazol-4-yl)-4-chlorophenoxy]-5-chloro-2-fluoro-N-(1,3-thiazol-4-yl)benzenesulfonamide or a pharmaceutically acceptable salt thereof.

In another aspect the invention provides a method of treating a disease where a URAT-1 inhibitor is indicated which comprises administering an effective amount of 4-[2-(5-amino-1H-pyrazol-4-yl)-4-chlorophenoxy]-5-chloro-2-fluoro-N-(1,3-thiazol-4-yl)benzenesulfonamide or a pharmaceutically acceptable salt thereof.

Surprisingly, the tosylate salt of 4-[2-(5-amino-1H-pyrazol-4-yl)-4-chlorophenoxy]-5-chloro-2-fluoro-N-(1,3-thiazol-4-yl)benzenesulfonamide has been found to have a number of unexpected properties making it especially suitable for the preparation of pharmaceutically acceptable formulations. The tosylate salt shows improved chemical stability over the free base, in particular with regard to formulation and storage. It can also be made in crystalline form, affording better solid form stability than the free base. Surprisingly the tosylate salt showed greater stability with regard to disassociation than other salts, and also demonstrated good aqueous solubility.

Accordingly, in another aspect the invention provides the tosylate salt of 4-[2-(5-amino-1H-pyrazol-4-yl)-4-chlorophenoxy]-5-chloro-2-fluoro-N-(1,3-thiazol-4-yl)benzenesulfonamide.

In one embodiment the tosylate salt of 4-[2-(5-amino-1H-pyrazol-4-yl)-4-chlorophenoxy]-5-chloro-2-fluoro-N-(1,3-thiazol-4-yl)benzenesulfonamide is a crystalline solid.

In a further embodiment the crystalline solid is characterized by a powder X-ray diffraction (PXRD) pattern which shows three, four, five or six of the characteristic two-theta (2θ) peaks selected from the set of peaks defined by tables 4 and 4a that follow, by using CuKalpha1 X-ray radiation (wavelength=1.5406 Å).

In a further embodiment, the crystalline solid is characterized by a PXRD pattern which shows any three, four, five or six of the characteristic 2θ peaks selected from the group consisting of: 9.0, 9.3, 10.0, 10.7, 11.6, 12.5, 12.9, 13.2, 13.8, 14.4, 16.0, 16.6, 17.5, 17.8, 18.1, 21.4 and 23.4° (+/−0.2° 2θ), more preferably from the group consisting of 9.0, 9.3, 10.0, 10.7, 11.6, 12.9, 13.2, 16.0, 16.6, 17.5, 17.8, 18.1, 21.4 and 23.4° (+/−0.2° 2θ), most preferably from the group consisting of 11.6, 12.9, 16.0, 17.5, 17.8, 18.1° (+/−0.2° 2θ) by using CuKalpha1 X-ray radiation (wavelength=1.5406 Å).

In a further embodiment the crystalline solid is characterized by a powder X-ray diffraction (PXRD) pattern which shows main two-theta (2θ) peaks at 9.0, 10.7, 16.0, 21.4 and 23.4° (+/−0.1° 2θ) by using CuKalpha1 X-ray radiation (wavelength=1.540562 Å).

4-[2-(5-Amino-1H-pyrazol-4-yl)-4-chlorophenoxy]-5-chloro-2-fluoro-N-(1,3-thiazol-4-yl)benzenesulfonamide or a pharmaceutically acceptable salt thereof, such as the tosylate salt, will generally be administered as a formulation in association with one or more pharmaceutically acceptable excipients. The term ‘excipient’ is used herein to describe any ingredient other than the aforementioned benzenesulfonamide. The choice of excipient will to a large extent depend on factors such as the particular mode of administration, the effect of the excipient on solubility and stability, and the nature of the dosage form.

In another aspect the invention provides a pharmaceutical composition comprising the tosylate salt of 4-[2-(5-amino-1H-pyrazol-4-yl)-4-chlorophenoxy]-5-chloro-2-fluoro-N-(1,3-thiazol-4-yl)benzenesulfonamide together with one or more pharmaceutically acceptable excipients.

Pharmaceutical compositions suitable for the delivery of 4-[2-(5-Amino-1H-pyrazol-4-yl)-4-chlorophenoxy]-5-chloro-2-fluoro-N-(1,3-thiazol-4-yl)benzenesulfonamide or a pharmaceutically acceptable salt thereof, such as the tosylate salt, and methods for their preparation will be readily apparent to those skilled in the art. Such compositions and methods may be found, for example, in “Remington's Pharmaceutical Sciences”, 19th Edition (Mack Publishing Company, 1995).

Suitable modes of administration include oral, parenteral, topical, inhaled/intranasal, rectal/intravaginal, and ocular/aural administration.

Formulations suitable for the aforementioned modes of administration may be formulated to be immediate and/or modified release. Modified release formulations include delayed-, sustained-, pulsed-, controlled-, targeted and programmed release.

4-[2-(5-Amino-1H-pyrazol-4-yl)-4-chlorophenoxy]-5-chloro-2-fluoro-N-(1,3-thiazol-4-yl)benzenesulfonamide or a pharmaceutically acceptable salt thereof, such as the tosylate salt, may be administered orally. Oral administration may involve swallowing, so that the drug enters the gastrointestinal tract, or buccal or sublingual administration may be employed by which the drug enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films, ovules, sprays, liquid formulations and buccal/mucoadhesive patches.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

4-[2-(5-Amino-1H-pyrazol-4-yl)-4-chlorophenoxy]-5-chloro-2-fluoro-N-(1,3-thiazol-4-yl)benzenesulfonamide or a pharmaceutically acceptable salt thereof, such as the tosylate salt, may also be used in fast-dissolving, fast-disintegrating dosage forms such as those described in Expert Opinion in Therapeutic Patents, 11 (6), 981-986, by Liang and Chen (2001).

For tablet dosage forms, depending on dose, 4-[2-(5-amino-1H-pyrazol-4-yl)-4-chlorophenoxy]-5-chloro-2-fluoro-N-(1,3-thiazol-4-yl)benzenesulfonamide or a pharmaceutically acceptable salt thereof, such as the tosylate salt, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet. Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant. Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. The formulation of tablets is discussed in “Pharmaceutical Dosage Forms: Tablets”, Vol. 1, by H. Lieberman and L. Lachman (Marcel Dekker, New York, 1980).

Suitable modified release formulations for the purposes of the invention are described in U.S. Pat. No. 6,106,864. Details of other suitable release technologies such as high energy dispersions and osmotic and coated particles are to be found in “Pharmaceutical Technology On-line”, 25(2), 1-14, by Verma et al (2001). The use of chewing gum to achieve controlled release is described in WO 00/35298.

4-[2-(5-Amino-1H-pyrazol-4-yl)-4-chlorophenoxy]-5-chloro-2-fluoro-N-(1,3-thiazol-4-yl)benzenesulfonamide or a pharmaceutically acceptable salt thereof, such as the tosylate salt, may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of 4-[2-(5-amino-1H-pyrazol-4-yl)-4-chlorophenoxy]-5-chloro-2-fluoro-N-(1,3-thiazol-4-yl)benzenesulfonamide or a pharmaceutically acceptable salt thereof, such as the tosylate salt, used in the preparation of parenteral solutions may be increased by the use of appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted and programmed release.

4-[2-(5-Amino-1H-pyrazol-4-yl)-4-chlorophenoxy]-5-chloro-2-fluoro-N-(1,3-thiazol-4-yl)benzenesulfonamide or a pharmaceutically acceptable salt thereof, such as the tosylate salt, may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

4-[2-(5-Amino-1H-pyrazol-4-yl)-4-chlorophenoxy]-5-chloro-2-fluoro-N-(1,3-thiazol-4-yl)benzenesulfonamide or a pharmaceutically acceptable salt thereof, such as the tosylate salt, can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the drug product, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

Suitable flavours, such as menthol and levomenthol, or sweeteners, such as saccharin or saccharin sodium, may be added to those formulations of the invention intended for inhaled/intranasal administration.

In the case of dry powder inhalers and aerosols, the dosage unit is determined by means of a valve which delivers a metered amount. Units in accordance with the invention are typically arranged to administer a metered dose or “puff” containing from 1 μg to 100 mg of the compound of list (I). The overall daily dose will typically be in the range 1 μg to 200 mg which may be administered in a single dose or, more usually, as divided doses throughout the day.

4-[2-(5-Amino-1H-pyrazol-4-yl)-4-chlorophenoxy]-5-chloro-2-fluoro-N-(1,3-thiazol-4-yl)benzenesulfonamide or a pharmaceutically acceptable salt thereof, such as the tosylate salt, may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

4-[2-(5-Amino-1H-pyrazol-4-yl)-4-chlorophenoxy]-5-chloro-2-fluoro-N-(1,3-thiazol-4-yl)benzenesulfonamide or a pharmaceutically acceptable salt thereof, such as the tosylate salt, may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

For administration to human patients, the total daily dose of 4-[2-(5-amino-1H-pyrazol-4-yl)-4-chlorophenoxy]-5-chloro-2-fluoro-N-(1,3-thiazol-4-yl)benzenesulfonamide or a pharmaceutically acceptable salt thereof, such as the tosylate salt, is typically in the range 1 mg to 10 g, such as 10 mg to 1 g, for example 25 mg to 500 mg depending, of course, on the mode of administration and efficacy. The total daily dose may be administered in single or divided doses and may, at the physician's discretion, fall outside of the typical range given herein, depending on the age, weight and response of the particular patient.

4-[2-(5-amino-1H-pyrazol-4-yl)-4-chlorophenoxy]-5-chloro-2-fluoro-N-(1,3-thiazol-4-yl)benzenesulfonamide or a pharmaceutically acceptable salt thereof, such as the tosylate salt, may be usefully combined with another pharmacologically active compound, or with two or more other pharmacologically active compounds, for the treatment of gout. Such combinations offer the possibility of significant advantages, including patient compliance, ease of dosing and synergistic activity.

In the combinations that follow the compound of the invention may be administered simultaneously, sequentially or separately in combination with the other therapeutic agent or agents.

4-[2-(5-Amino-1H-pyrazol-4-yl)-4-chlorophenoxy]-5-chloro-2-fluoro-N-(1,3-thiazol-4-yl)benzenesulfonamide, or a pharmaceutically acceptable salt thereof, such as the tosylate salt, may be administered in combination with one or more agents selected from:
an anti-inflammatory drug such as an NSAID (e.g. celecoxib), colchicine or a steroid;
a xanthine oxidase inhibitor (e.g. allopurinol, febuxostat or tisopurine) or a purine nucleoside phosphorylase (PNP) inhibitor (e.g. ulodesine);
a uricase (e.g. pegloticase or rasburicase); or a
a uricosuric, such as an agent that inhibits the transporters responsible for renal reabsorption of uric acid back into the blood, such as benziodarone, isobromindione, probenecid and sulphinpyrazone, or a URAT-1 inhibitor (e.g. benzbromarone).

It is to be appreciated that all references herein to treatment include curative, palliative and prophylactic treatment.

4-[2-(5-Amino-1H-pyrazol-4-yl)-4-chlorophenoxy]-5-chloro-2-fluoro-N-(1,3-thiazol-4-yl)benzenesulfonamide may be prepared by any method known in the art for the preparation of compounds of analogous structure and, in particular, by the specific methods described in WO2010/079443, such as that set out in Example 788.

The invention is illustrated by the following non-limiting examples.

### EXAMPLE 1

### Preparation of 4-[2-(5-amino-1H-pyrazol-4-yl)-4-chlorophenoxy]-5-chloro-2-fluoro-N-(1,3-thiazol-4-yl)benzenesulfonamide tosylate

To 4-[2-(5-amino-1H-pyrazol-4-yl)-4-chlorophenoxy]-5-chloro-2-fluoro-N-(1,3-thiazol-4-yl)benzenesulfonamide (example 788 WO2010/079443, 36.75 g, 73.45 mmol) in ethyl acetate (20 mL/g, 735 mL) was added methanol (3 mL/g, 110.25 mL) and the mixture heated to 50° C. A solution of p-toluenesulfonic acid monohydrate (13.27 g, 69.77 mmol) in methanol (2 mL/g, 73.50 mL) was added to the reaction mixture over 6 minutes via dropping funnel followed by addition of further methanol (1 mL/g, 36.75 mL). The reaction mixture was cooled to room temperature, filtered under vacuum and the solid washed with ethyl acetate:methanol (9:1, 2×37 mL). The solid was dried in vacuo at 50° C. overnight to provide the title compound as a free flowing off-white solid (43.99 g, 65.41 mmol, 89%).

Details of the spectroscopic analysis of the tosylate salt of 4-[2-(5-amino-1H-pyrazol-4-yl)-4-chlorophenoxy]-5-chloro-2-fluoro-N-(1,3-thiazol-4-yl)benzenesulfonamide follow:

### Infrared (IR) Spectroscopy

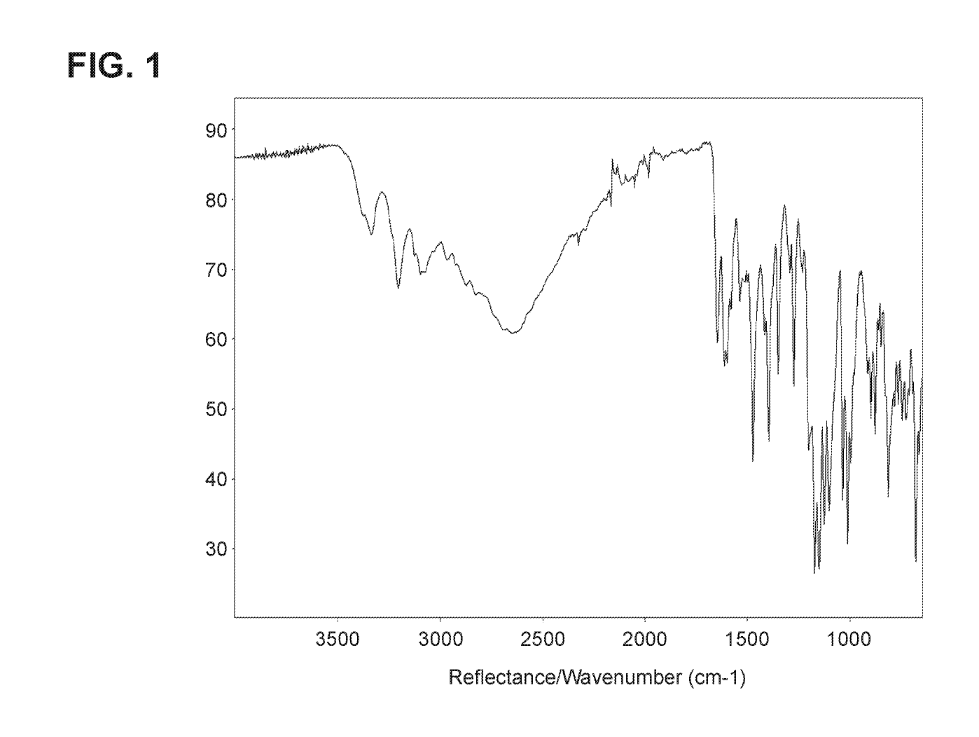

The infrared absorption spectrum was recorded using single reflection Attenuated Total Reflectance (ATR). The spectrum was acquired at 4 cm-1 resolution using a ThermoNicolet Avatar 360 FT IR spectrometer and a Smart Golden Gate™ accessory. This approach required no sample preparation. The spectrum is shown in FIG. 1.

### Mass Spectrometry (MS)

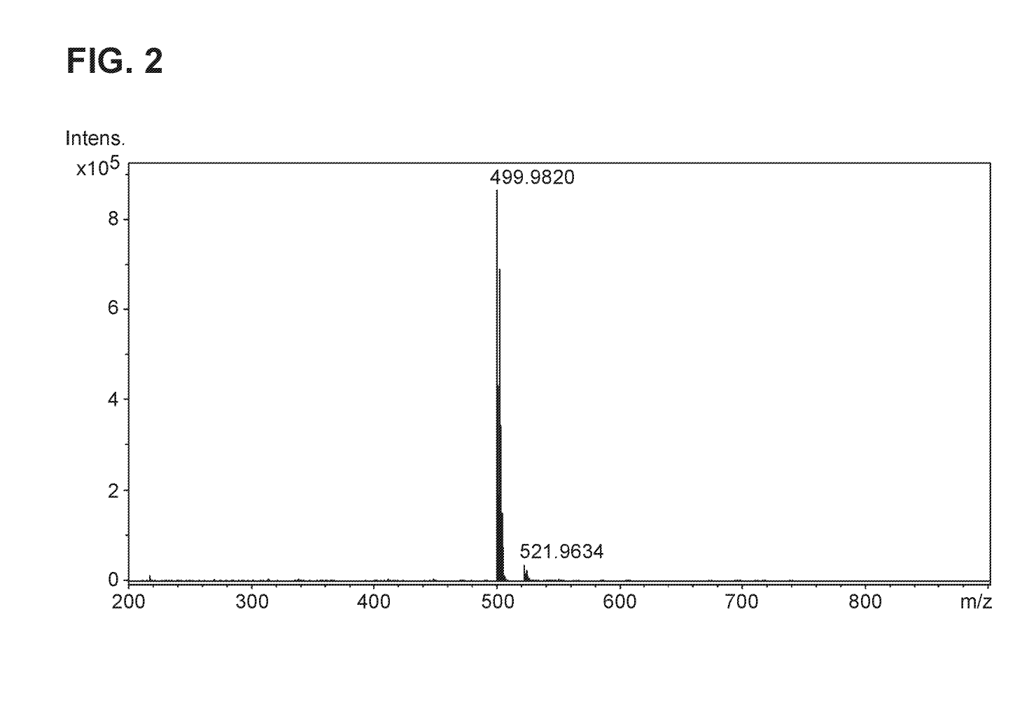

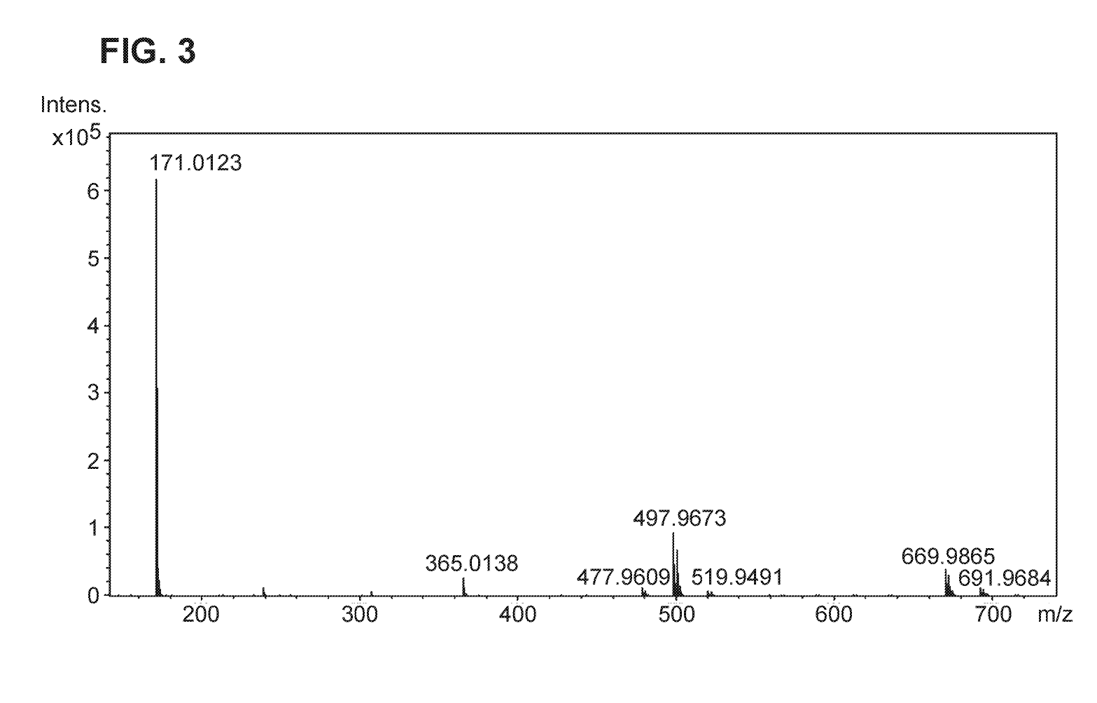

The full scan mass spectra are presented in FIG. 2 and FIG. 3 and were obtained by electrospray positive (ES+) and negative (ES−) ionization respectively. Data were recorded using a Bruker MaX is Quadrupole Time of Flight mass spectrometer fitted with an electrospray source. Internal calibration was performed using a sodium formate solution, which gave an observed maximum mass deviation of 0.2 mDa (ES+) and 0.3 mDa (ES−) over the mass range m/z 113 to m/z 997.

The accurate mass measurement, theoretical monoisotopic mass and molecular formula of the observed adduct and fragment ions for the ES+ and ES− data are shown in Tables 1 and 2 respectively. Corresponding mass spectra are show in FIGS. 2 and 3 respectively.

TABLE 1
ES+ Accurate Mass Data

| | | | Theo- | |
|---|---|---|---|---|
| | | | retical | |
| Mass | | | Mono- | Mass |
| Measure- | | | isotopic | Devia- |
| ment | Ion | | Mass | tion |
| (m/z) | Assignment | Formula of Ion | (m/z) | (mDa) |
| 499.9820 | [M + H]+ | C₁₈H₁₃Cl₂FN₅O₃S₂+ | 499.9815 | 0.5 |
| 521.9634 | [M + Na]+ | C₁₈H₁₂Cl₂FN₅O₃S₂Na+ | 521.9635 | 0.1 |

TABLE 2
ES− Accurate Mass Data

| Mass | | | Theoretical | Mass |
|---|---|---|---|---|
| Measurement | Ion | | Monoisotopic | Deviation |
| (m/z) | Assignment | Formula of Ion | Mass (m/z) | (mDa) |
| 171.0123 | [M − H]− for para | C₇H₇O₃S− | 171.0121 | 0.2 |
| | toluenesulfonic | | | |
| | acid | | | |
| 365.0138 | [2M + Na − 2H]− | C₁₄H₁₄O₆S₂Na− | 365.0135 | 0.3 |
| | for para- | | | |
| | toluenesulfonic | | | |
| | acid | | | |
| 477.9609 | [M − H − HF]− | C₁₈H₁₀Cl₂N₅O₃S₂− | 477.9608 | 0.1 |
| 497.9673 | [M − H]− | C₁₈H₁₁Cl₂FN₅O₃S₂− | 497.9670 | 0.3 |
| 519.9491 | [M + Na − 2H]− | C₁₈H₁₀Cl₂FN₅O₃S₂Na− | 519.9489 | 0.2 |
| 669.9865 | [M+ para- | C₂₅H₁₉Cl₂FN₅O₆S₃− | 669.9864 | 0.1 |
| | toluenesulfonic | | | |
| | acid H]− | | | |
| 691.9684 | [M+ para- | C₂₅H₁₈Cl₂FN₅O₆S₃Na− | 691.9683 | 0.1 |
| | toluenesulfonic | | | |
| | acid + Na − 2H]− | | | |

### Nuclear Magnetic Resonance (NMR) Spectroscopy

The proton (¹H) NMR spectrum was acquired in solution in DMSO d₆. Data were obtained at 30° C. on a Bruker AVANCE III 600 MHz NMR spectrometer equipped with a triple resonance cryogenic probe tuned for protons at 599.77 MHz. The spectrum was referenced to DMSO d₅ (2.50 ppm).

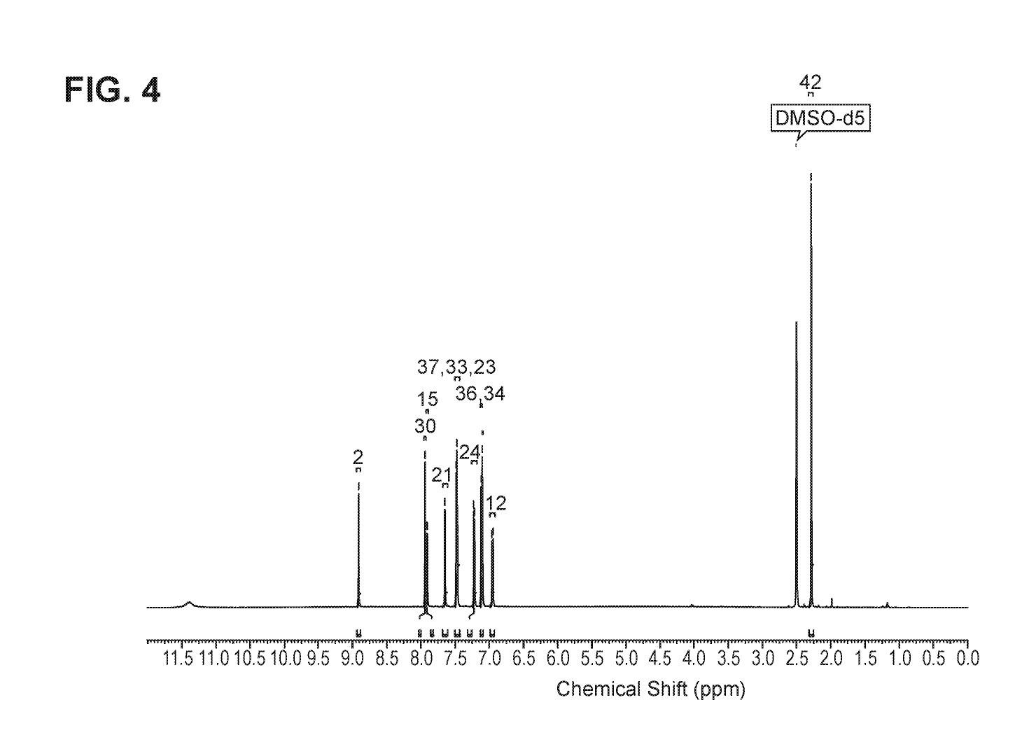

The ¹H NMR spectrum, shown in FIG. 4 and labeled with reference to the structure below, reveals the presence of 12 aromatic and three aliphatic (one CH₃ group) protons. ¹H chemical shift assignments are summarized in Table 3.

TABLE 3
1H Assignments in DMSO d6

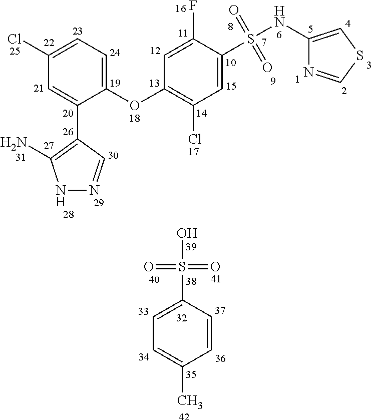

| Atom | ¹H Chemical | ¹H | No. of | |
|---|---|---|---|---|
| Number | Shift (ppm) | Multiplicity | ¹H | J (Hz) |
| 42 | 2.29 | singlet | 3 | — |
| 12 | 6.95 | doublet | 1 | 10.8 |
| 4 | 7.10 | doublet | 1 | 2.2 |
| 34, 36 | 7.11 | doublet | 2 | 7.9 |
| 24 | 7.22 | doublet | 1 | 8.7 |
| 37, 33, 23 | 7.43-7.50 | multiplet | 3 | — |
| 21 | 7.65 | doublet | 1 | 2.6 |
| 15 | 7.91 | doublet | 1 | 7.1 |
| 30 | 7.94 | singlet | 1 | — |
| 2 | 8.91 | doublet | 1 | 2.2 |

### Ultraviolet/Visible (UV/Vis) Spectrophotometry

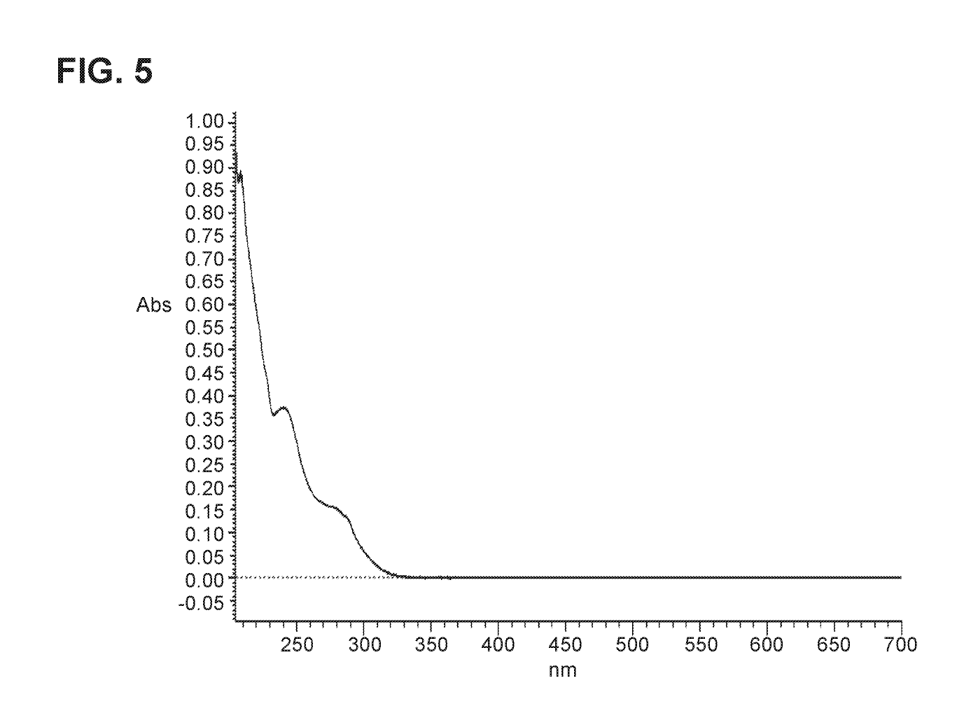

The UV/Visible spectrum was acquired using a Hitachi U-3000 spectrophotometer in methanol at a concentration of 1.09 mg/100 mL and is shown in FIG. 5. Two λₘₐₓ are observed at 281 and at 240 nm.

### Characterisation of the 4-[2-(5-amino-1H-pyrazol-4-yl)-4-chlorophenoxy]-5-chloro-2-fluoro-N-(1,3-thiazol-4-yl)benzenesulfonamide tosylate by PXRD

The powder X-ray diffraction pattern of 4-[2-(5-amino-1H-pyrazol-4-yl)-4-chlorophenoxy]-5-chloro-2-fluoro-N-(1,3-thiazol-4-yl)benzenesulfonamide tosylate was determined using a Bruker-AXS Ltd. D4 ENDEAVOR powder X-ray diffractometer fitted with an automatic sample changer, a theta-theta goniometer geometry, automatic beam divergence slit and a PSD Vantec-1 detector. The sample was prepared for analysis by mounting onto a low background silicon wafer specimen mount with a 0.5 mm cavity. The specimen was rotated whilst being irradiated with copper Kα₁ X-rays (wavelength=1.5406 Ångstroms) with the X-ray tube operated at 35 kV/40 mA. The analysis was in continuous mode set for data acquisition at 0.2 second count per 0.018° step size over a two theta range of 2° to 55° at room temperature. Peak search was carried out using the threshold and width parameters set to 1 and 0.3, respectively, within the Eva software released by Bruker-AXS. The instrument calibration was verified using a corundum reference standard (NIST: SRM 1976 XRD flat plate intensity standard).

Due to differences in instruments, samples, and sample preparation, the peak values are reported herein with an estimate of variability for the peak values. This is common practice in the solid-state chemical arts because of the variation inherent in peak values. A typical variability of the 2θ x-axis value for powder x-ray diffraction is on the order of plus or minus 0.2° 2θ.

Variability in peak intensity is a result of how individual crystals are oriented in the sample container with respect to the external X-ray source (known as “preferred orientation”). This orientation effect does not provide structural information about the crystal.

Additionally, one skilled in the art would also recognize that the intensities of characteristic peaks described above would change when the crystalline material of the present invention is mixed or diluted with additional components, such as pharmaceutical excipients. For this reason, the person skilled in the art would appreciate that the PXRD method described above may have to be optimized slightly to enable detection of the characteristic peaks within a mixture of components. This optimization may include the use of a more intense X-ray source (wavelength=1.5406 Ångstroms), a slightly different step size, or a slightly different step time.

The skilled person will also appreciate that measurements using a different wavelength will result in different shifts according to the Bragg equation—nλ=2d sin θ. Such further PXRD patterns generated by use of alternative wavelengths are considered to be alternative representations of the PXRD patterns of the crystalline materials of the present invention and as such are within the scope of the present invention.

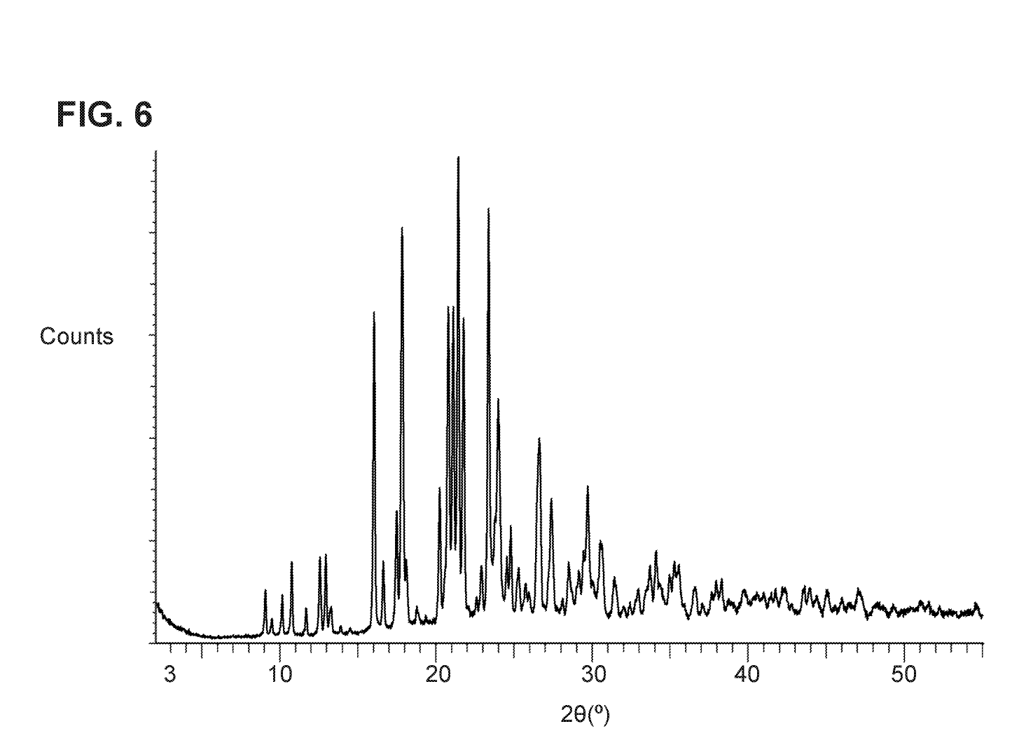

The PXRD pattern is shown in FIG. 6. The main 2θ peak positions and relative intensities are listed in Tables 4 and 4a.

TABLE 4
Characteristic diffraction peaks of 4-[2-(5-amino-1H-pyrazol-4-yl)-4-
chlorophenoxy]-5-chloro-2-fluoro-N-(1,3-thiazol-4-yl)benzenesulfonamide
tosylate (±0.2° 2θ) with relative intensity cut-off ≧10%

| Angle (°2θ) | Intensity % |
|---|---|
| 9.0 | 10.8 |
| 10.7 | 16.6 |
| 12.5 | 17.6 |
| 12.9 | 18.1 |
| 16.0 | 68.0 |
| 16.6 | 16.7 |
| 17.5 | 27.0 |
| 17.8 | 85.4 |
| 18.1 | 16.3 |
| 20.2 | 31.8 |
| 20.8 | 69.1 |
| 21.1 | 69.1 |
| 21.4 | 100.0 |
| 21.7 | 66.7 |
| 22.9 | 15.8 |
| 23.4 | 89.3 |
| 24.0 | 50.1 |
| 24.5 | 17.7 |
| 24.8 | 24.0 |
| 25.3 | 15.4 |
| 25.7 | 12.1 |
| 26.0 | 10.4 |
| 26.6 | 42.0 |
| 27.4 | 29.6 |
| 28.5 | 16.5 |
| 29.2 | 14.4 |
| 29.5 | 18.9 |
| 29.7 | 32.1 |
| 30.0 | 12.5 |
| 30.6 | 20.9 |
| 31.4 | 13.5 |
| 32.9 | 10.8 |
| 33.5 | 10.8 |
| 33.7 | 15.8 |
| 34.1 | 18.9 |
| 34.4 | 12.1 |
| 35.0 | 13.9 |
| 35.3 | 16.6 |
| 35.6 | 16 |
| 36.6 | 11.5 |
| 37.7 | 10.3 |
| 38.0 | 12.7 |
| 38.3 | 12.7 |
| 39.7 | 10.8 |
| 41.0 | 10.3 |
| 41.5 | 10.3 |
| 41.8 | 11.1 |
| 42.4 | 11.3 |
| 43.6 | 11.1 |
| 44.0 | 11.2 |
| 45.1 | 10.9 |
| 47.1 | 11.2 |

TABLE 4a
Characteristic diffraction peaks of 4-[2-(5-amino-1H-pyrazol-4-yl)-4-
chlorophenoxy]-5-chloro-2-fluoro-N-(1,3-thiazol-4-yl)benzenesulfonamide
tosylate (±0.2° 2θ) with less than 10% relative intensity.

| Angle (°2θ) | Intensity % |
|---|---|
| 9.3 | 3.6 |
| 10.0 | 5.7 |
| 11.6 | 6.9 |
| 13.2 | 7.2 |
| 13.8 | 3.8 |
| 14.4 | 4.1 |

### EXAMPLE 2

### Preparation of 4-[2-(5-amino-1H-pyrazol-4-yl)-4-chlorophenoxy]-5-chloro-2-fluoro-N-(1,3-thiazol-4-yl)benzenesulfonamide spray dried dispersion (SDD)

Tetrahydrofuran (unstabilized, 14.5 kg) and water (0.76 kg) were added to a stainless-steel tank equipped with a top mount mixer. 4-[2-(5-Amino-1H-pyrazol-4-yl)-4-chlorophenoxy]-5-chloro-2-fluoro-N-(1,3-thiazol-4-yl)benzenesulfonamide (742.4 g) was then added to the solution and mixed for at least 1 hour until all solids had fully dissolved. Hydroxypropyl methylcellulose acetate succinate (medium grade granular, 1338.4 g) was added to the solution and mixed until fully dissolved. The solution was then spray dried under nitrogen gas using the conditions tabulated below.

| Process | Process Parameters | Target | | Target Range | |
|---|---|---|---|---|---|
| (A) Preheating | Nitrogen drying-gas flow | 1850 | g/min | 1550 to 2150 | g/min |
| | Dryer inlet temperature (Tᵢₙ) | 110° | C. | 100° C. to 120° C. | |
| (B) Warm-Up/ | Nitrogen drying-gas flow | 1850 | g/min | 1550 to 2150 | g/min |
| Shutdown | Tᵢₙ | 110° | C. | 100° C. to 120° C. | |
| | Dryer outlet temperature (Tₒᵤₜ) | 52° | C. | 47° C. to 57° C. | |
| | Solvent atomization | 600 | psi | 450 to 750 | psi |
| | pressure | | | | |
| | Solvent feed rate | 140 | g/min | 115 to 165 | g/min |
| (C) Feed-Solution | Nitrogen drying-gas flow | 1850 | g/min | 1550 to 2150 | g/min |
| Processing | Tᵢₙ | 110° | C. | 100° C. to 120° C. | |
| | Tₒᵤₜ | 45° | C. | 40° C. to 50° C. | |
| | Solution atomization | 500 | psi | 400 to 600 | psi |
| | pressure | | | | |
| | Solution feed rate | 155 | g/min | 130 to 180 | g/min |

NB: 95:5 (w/w %) of tetrahydrofuran (unstabilized):Water was used for system start up/shutdown.

The resulting SDD of 4-[2-(5-amino-1H-pyrazol-4-yl)-4-chlorophenoxy]-5-chloro-2-fluoro-N-(1,3-thiazol-4-yl)benzenesulfonamide was then tray dried in a convection tray dryer at 40° C./50% relative humidity (RH) for a minimum of 6 hours, followed by a ramp up to 40° C./75°/ORH for an additional minimum of 25 hours.

The SDD was stored at 2 to 8° C. until required.

### EXAMPLE 3

### Preparation of Dispersions for Oral Administration

### (a) Using the tosylate salt of 4-[2-(5-Amino-1H-pyrazol-4-yl)-4-chlorophenoxy]-5-chloro-2-fluoro-N-(1,3-thiazol-4-yl)benzenesulfonamide

Methylcellulose vehicle (0.5% w/v) was prepared as follows. Water for irrigation (600 mL) was heated in a beaker to between 80° C. and 90° C. Methylcellulose (5 g) powder was added, with stirring, until the powder had fully dispersed. The dispersion was then transferred into an ice bath and cooled rapidly whilst adding chilled water for irrigation (400 mL) to give a clear solution.
The dispersion for oral administration was prepared by weighing the required quantity of 4-[2-(5-amino-1H-pyrazol-4-yl)-4-chlorophenoxy]-5-chloro-2-fluoro-N-(1,3-thiazol-4-yl)benzenesulfonamide tosylate (from 10 mg to 2400 mg) into an appropriately sized glass amber dosing bottle and adding a volume of vehicle (0.5% (w/v) methylcellulose). The volume of vehicle added was dependant on dose: 15 mL for doses of drug in the range 10 mg to <30 mg; and 50 mL for doses of drug in the range 30 mg to 2400 mg, so that the drug concentration was in the range 0.6 to 50 mg/mL.
The dispersion was stored at 2 to 8° C. and administered within 72 hours directly from the dosing containers. After administration the glass dosing bottle was rinsed with two approximate equal aliquots of drinking water such that the total volume administered, including dosing volume, was 240 mL.

### (b) Using the SDD of 4-[2-(5-Amino-1H-pyrazol-4-yl)-4-chlorophenoxy]-5-chloro-2-fluoro-N-(1,3-thiazol-4-yl)benzenesulfonamide

Methylcellulose vehicle 0.5% (w/v) was prepared using the procedure set out in Example 3(a) above.
The dispersion for oral administration was prepared by weighing the required quantity of 4-[2-(5-amino-1H-pyrazol-4-yl)-4-chlorophenoxy]-5-chloro-2-fluoro-N-(1,3-thiazol-4-yl)benzenesulfonamide SDD into an appropriately sized glass amber dosing bottle and adding a volume of vehicle (0.5% (w/v) Methylcellulose). The volume of vehicle added was dependent on dose: 20 mL to 100 mL over the dose range 10 mg to 2400 mg, so that the drug concentration was in the range 0.6 to 50 mg/mL.
The dispersion was stored at 2 to 8° C. and administered within 72 hours directly from the dosing containers. After administration the glass dosing bottle was rinsed with two approximate equal aliquots of drinking water such that the total volume administered, including dosing volume, was 240 mL.

### Biological Activity

The ability of 4-[2-(5-amino-1H-pyrazol-4-yl)-4-chlorophenoxy]-5-chloro-2-fluoro-N-(1,3-thiazol-4-yl)benzenesulfonamide and pharmaceutically acceptable salts thereof, such as the tosylate salt, to lower blood uric acid levels was demonstrated in the following experiments. Uric acid measurements were performed using a commercial colorimetric assay kit (Beckman Coulter).

### EXAMPLE 4

### Single Dose Study

### A Double-Blind, Randomized, Placebo-Controlled, Crossover Study in Six Cohorts of Healthy Subjects

Single doses of 4-[2-(5-amino-1H-pyrazol-4-yl)-4-chlorophenoxy]-5-chloro-2-fluoro-N-(1,3-thiazol-4-yl)benzenesulfonamide SDD (‘SDD’) dispersion for oral administration, ranging from 10 mg to 2400 mg, were investigated. In addition, single doses of 4-[2-(5-amino-1H-pyrazol-4-yl)-4-chlorophenoxy]-5-chloro-2-fluoro-N-(1,3-thiazol-4-yl)benzenesulfonamide tosylate (‘TS’) dispersion for oral administration, ranging from 200 mg to 1000 mg, were also investigated.

The following doses were investigated and all doses were given in the fasted state with the exception of 200 mg and 1000 mg SDD dispersions, which were given in both the fasted state and after a high-fat meal (‘fed’).

Cohort 1: 10 mg SDD, 100 mg SDD, 300 mg SDD, 200 mg TS, placebo

Cohort 2: 30 mg SDD, 300 mg SDD, 200 mg SDD (fed), placebo

Cohort 3: 100 mg SDD, 200 mg SDD, 300 mg SDD, placebo

Cohort 4: 450 mg SDD, 600 mg SDD, 800 mg SDD, 1000 mg SDD, placebo

Cohort 5: 600 mg TS, 1000 mg TS, 1000 mg SDD (fed)

Cohort 6: 1250 mg SDD, 1600 mg SDD, 2000 mg SDD, 2400 mg SSD, placebo

A total of 61 subjects (all male) participated and all received at least one dose of SDD or TS dispersion for oral administration.

Mean data of uric acid levels per dose group are given as follows:
SDD fasted: Table 5
SDD fed and fasted Table 6
TS Table 7

TABLE 5

| Dose (mg) | Time (hours) | Mean uric acid (mg/dl) |
|---|---|---|
| 0 | 0 | 5.54 |
| 0 | 48 | 5.68 |
| 10 | 0 | 5.59 |
| 10 | 48 | 5.04 |
| 30 | 0 | 5.64 |
| 30 | 48 | 5.85 |
| 100 | 0 | 5.70 |
| 100 | 48 | 5.46 |
| 200 | 0 | 5.62 |
| 200 | 48 | 5.10 |
| 300 | 0 | 5.63 |
| 300 | 48 | 4.77 |
| 450 | 0 | 5.78 |
| 450 | 48 | 4.47 |
| 600 | 0 | 6.00 |
| 600 | 48 | 4.55 |
| 800 | 0 | 5.88 |
| 800 | 48 | 4.15 |
| 1000 | 0 | 5.87 |
| 1000 | 48 | 3.73 |
| 1250 | 0 | 5.81 |
| 1250 | 48 | 3.65 |
| 1600 | 0 | 5.52 |
| 1600 | 48 | 3.64 |
| 2000 | 0 | 5.40 |
| 2000 | 48 | 3.55 |

TABLE 6

| Dose (mg) | Time (hours) | Mean uric acid (mg/dL) |
|---|---|---|
| 0 | 0 | 5.59 |
| 0 | 48 | 5.71 |
| 10 | 0 | 5.59 |
| 10 | 48 | 5.04 |
| 30 | 0 | 5.64 |
| 30 | 48 | 5.85 |
| 100 | 0 | 5.70 |
| 100 | 48 | 5.46 |
| 200 | 0 | 5.62 |
| 200 | 48 | 5.10 |
| 200 Fed | 0 | 5.92 |
| 200 Fed | 48 | 4.92 |
| 300 | 0 | 5.63 |
| 300 | 48 | 4.77 |
| 450 | 0 | 5.78 |
| 450 | 48 | 4.47 |
| 600 | 0 | 6.00 |
| 600 | 48 | 4.55 |
| 800 | 0 | 5.88 |
| 800 | 48 | 4.15 |
| 1000 | 0 | 5.87 |
| 1000 | 48 | 3.73 |
| 1000 Fed | 0 | 6.41 |
| 1000 Fed | 48 | 4.13 |
| 1250 | 0 | 5.81 |
| 1250 | 48 | 3.65 |
| 1600 | 0 | 5.52 |
| 1600 | 48 | 3.64 |
| 2000 | 0 | 5.48 |
| 2000 | 48 | 3.82 |
| 2400 | 0 | 5.68 |
| 2400 | 48 | 3.60 |

TABLE 7

| Dose (mg) | Time (hours) | Mean uric acid (mg/dL) |
|---|---|---|
| 200 | 0 | 5.92 |
| 200 | 48 | 5.43 |
| 600 | 0 | 6.21 |
| 600 | 48 | 5.04 |
| 1000 | 0 | 6.40 |
| 1000 | 48 | 4.86 |

A dose related decrease in uric acid in blood was noted at 48 hours postdose. Although values generally remained within the normal range (3.5 to 7.2 mg/dL) at doses of 10 to 1000 mg, at doses of 1250 to 2400 mg the uric acid level reductions were more marked, with at least half of the subjects having values below the lower limit of normal (LLN) at 48 hours postdose. All predose and follow-up values were above the LLN. All of the subjects on placebo (n=42) had uric acid values within the normal range, except for one subject who was just below the LLN at 48 h postdose.

### EXAMPLE 5

### Multiple Dose Study

### A Double-Blind, Randomized, Placebo-Controlled, Study in Healthy Subjects

Multiple oral doses of 100 mg twice daily (BID), 300 mg BID, and 600 mg BID of 4-[2-(5-amino-1H-pyrazol-4-yl)-4-chlorophenoxy]-5-chloro-2-fluoro-N-(1,3-thiazol-4-yl)benzenesulfonamide SDD dispersion for oral administration, and placebo, were investigated for 14 days. Subjects were fasted overnight prior to the morning doses and for at least 2 hours prior to the evening doses. Food was withheld for at least 2 hours postdose.

A total of 30 subjects (all male) were enrolled in the study and 27 subjects completed the study (three subjects were withdrawn due to adverse events during treatment with 600 mg BID 4-[2-(5-amino-1H-pyrazol-4-yl)-4-chlorophenoxy]-5-chloro-2-fluoro-N-(1,3-thiazol-4-yl)benzenesulfonamide).

Mean data of uric acid levels per dose group are given in Table 8 and FIG. 7.

TABLE 8

| Dose (mg) | Time (hours) | Mean uric acid (mg/dL) |
|---|---|---|
| 0 | 0 | 5.47 |
| 0 | 4 | 5.40 |
| 0 | 7 | 4.08 |
| 0 | 10 | 5.40 |
| 0 | 14 | 5.23 |
| 0 | 16 | 5.12 |
| 0 | 26 | 5.33 |
| 100 | 0 | 4.85 |
| 100 | 4 | 3.91 |
| 100 | 7 | 3.38 |
| 100 | 10 | 4.13 |
| 100 | 14 | 4.04 |
| 100 | 16 | 5.05 |
| 100 | 26 | 5.30 |
| 300 | 0 | 5.88 |
| 300 | 4 | 3.44 |
| 300 | 7 | 3.50 |
| 300 | 10 | 3.73 |
| 300 | 14 | 3.64 |
| 300 | 16 | 4.66 |
| 300 | 26 | 6.73 |
| 600 | 0 | 5.56 |
| 600 | 4 | 2.31 |
| 600 | 7 | 2.45 |
| 600 | 10 | 2.74 |
| 600 | 14 | 2.56 |
| 600 | 16 | 4.34 |
| 600 | 26 | 6.43 |

A dose related decrease in uric acid in blood was apparent by Day 4 (first postdose assessment) with the lowest mean values occurring on Days 4 or 7. Five of 8 subjects had uric acid values below the LLN on Day 7 at 100 mg BID (actual range for subjects with low uric acid was 2.6 to 3.4 mg/dL; one subject was below the limit at baseline; actual value 2.7 mg/dL) and at 300 mg (actual range for subjects with low uric acid was 2.2 to 3.4 mg/dL). All subjects had uric acid values below the LLN on Days 4 and 7 of 600 mg BID dosing (actual range was <1.5 to 3.0 mg/dL). Values generally increased on Days 10 and 14 despite continued dosing, although all except 1 subject had returned to the normal range by Day 16 (2 days post-last dose). This remaining subject had the lowest uric value at baseline (4.7 mg/dL) with subsequent values of 2.2 mg/dL (Day 4), <1.5 mg/dL (Day 7), 1.6 mg/dL (Day 10), 3.1 mg/dL (Day 14) and 4.9 mg/dL at follow-up. All subjects receiving placebo (n=6) had uric acid concentrations within the normal range at all timepoints.

### EXAMPLE 6

### Multiple Dose Study

### A Double-Blind, Randomized, Placebo-Controlled, Study in Healthy Subjects and Elderly Subjects

Multiple oral doses of 4-[2-(5-amino-1H-pyrazol-4-yl)-4-chlorophenoxy]-5-chloro-2-fluoro-N-(1,3-thiazol-4-yl)benzenesulfonamide TS dispersion for oral administration, and placebo, were investigated for 14 days, as follows:
healthy subjects, 300 mg twice daily (BID,)
healthy subjects, 450 mg BID, and
elderly subjects, 300 mg BID.

Subjects were fasted overnight prior to the morning doses and for at least 2 hours prior to the evening doses. Food was withheld for at least 2 hours postdose.

A total of 49 subjects were enrolled in the study of which 39 received 4-[2-(5-amino-1H-pyrazol-4-yl)-4-chlorophenoxy]-5-chloro-2-fluoro-N-(1,3-thiazol-4-yl)benzenesulfonamide TS and 10 received placebo.

Mean blood uric acid level and urine excretion data, per dose group, are given in Table 9. FIG. 8 shows percent uric acid excreted in urine.

TABLE 9

| | | Mean uric acid | Fraction excreted |
|---|---|---|---|
| Dose (mg) | Time (days) | (mg/dL) | of uric acid (%) |
| 0 | 0 | 5.37 | NA |
| 0 | 3 | 5.33 | NA |
| 0 | 6 | 5.38 | NA |
| 0 | 8 | 5.61 | NA |
| 0 | 10 | 5.47 | NA |
| 0 | 14 | 5.39 | NA |
| 300 | 0 | 5.56 | NA |
| 300 | 1 | NA | 8.44 |
| 300 | 3 | 3.50 | NA |
| 300 | 6 | 3.23 | 17.41 |
| 300 | 8 | 3.54 | NA |
| 300 | 10 | 3.79 | 14.99 |
| 300 | 14 | 3.78 | 6.43 |
| 450 | 0 | 5.89 | NA |
| 450 | 1 | NA | 8.61 |
| 450 | 3 | 3.05 | NA |
| 450 | 6 | 3.00 | 19.81 |
| 450 | 8 | 3.21 | NA |
| 450 | 10 | 3.45 | 17.09 |
| 450 | 14 | 3.56 | 7.69 |
| 300 elderly | 0 | 5.43 | NA |
| 300 elderly | 1 | NA | 6.17 |
| 300 elderly | 3 | 3.41 | NA |
| 300 elderly | 6 | 2.99 | 13.79 |
| 300 elderly | 8 | 3.57 | NA |
| 300 elderly | 10 | 3.37 | 13.02 |
| 300 elderly | 14 | 3.59 | 7.66 |

Concentrations of uric acid in blood decreased following dosing with 4-[2-(5-amino-1H-pyrazol-4-yl)-4-chlorophenoxy]-5-chloro-2-fluoro-N-(1,3-thiazol-4-yl)benzenesulfonamide. All subjects had blood uric acid values above the LLN on Day 1. Subjects who received placebo had blood uric acid concentrations above the LLN throughout the study. The median blood uric acid concentrations fell below LLN on Day 3 for subjects who received 450 mg BID 4, and the median was below the LLN for all cohorts (300 mg and 450 mg BID) on Day 6. Blood uric acid concentrations returned to above the LLN for all subjects within 2 days of cessation of 4-[2-(5-amino-1H-pyrazol-4-yl)-4-chlorophenoxy]-5-chloro-2-fluoro-N-(1,3-thiazol-4-yl)benzenesulfonamide dosing (i.e. by Day 16).

Uric acid was also measured in urine collected over 24 hours prior to dosing on Day 1, and then on Days 6, 14 and 16. The percent excreted fraction of uric acid in urine was calculated and analyzed with a linear mixed effects model. A summary of these data is presented in FIG. 8. The data suggest that the excreted fraction of uric acid in urine increases during dosing with 4-[2-(5-amino-1H-pyrazol-4-yl)-4-chlorophenoxy]-5-chloro-2-fluoro-N-(1,3-thiazol-4-yl)benzenesulfonamide and returns to baseline by Day 16.

### EXAMPLE 7

### URAT-1 Inhibitor Activity

The potency of 4-[2-(5-amino-1H-pyrazol-4-yl)-4-chlorophenoxy]-5-chloro-2-fluoro-N-(1,3-thiazol-4-yl)benzenesulfonamide as an inhibitor of the URAT-1 transporter was determined as follows.

HEK293 cells were grown in medium consisting of Dulbecco's modified Eagle medium (DMEM) with L-GlutaMax (4.5 g of glucose per litre), supplemented with heat-inactivated foetal calf serum (10% v/v), 100 U/mL penicillin and 100 μg/mL streptomycin. The HEK cells were routinely cultured in 75 cm² tissue culture flasks in a humidified incubator at approximately 37° C. in approximately 95% air/5% CO₂. Near confluent HEK cell cultures were harvested by trypsinisation, re-suspended in culture medium and the process was repeated once or twice weekly to provide sufficient cells for use.

For uptake experiments, HEK293 cells were seeded onto poly-D-Lysine-coated 24-well plates at a density of 4×10⁵ cells per well. The cells were cultured for 1 day at approximately 37° C. in a humidified incubator containing approximately 5% CO₂ in air. Thereafter, cells were transfected with either pcDNA3.1/hygro/URAT1 (HEK-URAT1 cells) or pcDNA3.1/hygro (HEK-control cells) using Lipofectamine 2000 reagent. After approximately 24 hours at approximately 37° C. in a humidified incubator containing approximately 5% CO₂ in air, cells were used for experiments.

One day after transfection, culture medium was removed from the wells and the cells were pre-incubated with 0.2 mL of chlorine-free incubation medium (125 mM Na-gluconate, 4.8 mM K-gluconate, 1.3 mM Ca-gluconate, 1.2 mM KH₂PO₄, 1.2 mM MgSO₄, 5.6 mM D-glucose, 25 mM HEPES, pH 7.4), in the absence and presence of 4-[2-(5-amino-1H-pyrazol-4-yl)-4-chlorophenoxy]-5-chloro-2-fluoro-N-(1,3-thiazol-4-yl)benzenesulfonamide (0-30 μM), for 15 minutes at approximately 37° C. Thereafter, the incubation medium was removed and 0.2 mL of chlorine-free incubation medium containing [¹⁴C]-uric acid (20 μM) was added, in triplicate, in the absence and presence of 4-[2-(5-amino-1H-pyrazol-4-yl)-4-chlorophenoxy]-5-chloro-2-fluoro-N-(1,3-thiazol-4-yl)benzenesulfonamide (0-30 μM). Cells were incubated for 2 minutes at approximately 37° C. At the end of the incubation, the medium was aspirated and the monolayers rapidly rinsed twice with 1 mL of ice-cold incubation medium. Subsequently, the cells were solubilised in 0.5 mL of 0.5 N NaOH, and aliquots of cell lysate samples from each well were collected in scintillation vials. The concentrations of [¹⁴C]-uric acid were determined by liquid scintillation counting (LSC). The inhibition of [¹⁴C]-uric acid transport was also determined in the presence of the known inhibitor benzbromarone (30 μM). Final organic solvent were less than 1% (v/v).

The protein content of solubilised HEK cells was determined by the Bradford method using Bio-Rad Bradford Reagent with bovine serum albumin (BSA) as the protein standard (concentration range 0-1 mg/mL). The BSA solution or the solubilised cells were mixed with diluted dye reagent concentrate (Bio-Rad). The absorbance was measured at 595 nm after incubation at room temperature for 10 minutes.

The amount of radioactivity present in cell lysate samples was determined by liquid scintillation counting (LSC). Liquid scintillant (Hionic Fluor™) was added to all samples and radioactivity was determined by LSC on a Tri-Carb 3100TR liquid scintillation counter using QuantaSmart™ software in which all counts were converted to DPM using tSIE/AEC (transformed Spectral Index of external standards coupled to Automatic Efficiency Correction). Calibration procedures for the instruments are established at the testing facilities. All samples were counted for at least 2 minutes. Background values were measured with each sample sequence using liquid scintillant in the absence of sample. The accumulation (pmol/mg protein) of [¹⁴C]-Uric acid into HEK cells was calculated and the IC₅₀ values, defined as the concentration of inhibitor required for 50% inhibition of transport, was calculated using GraphPad Prism version 4.00 using the Hill equation.

### Data

The inhibition of uric acid uptake by 4-[2-(5-amino-1H-pyrazol-4-yl)-4-chlorophenoxy]-5-chloro-2-fluoro-N-(1,3-thiazol-4-yl)benzenesulfonamide measured by the methodology described above, normalised to the standard compound benzbromarone, is 3.54 uM.

1-24. (canceled)

25. A method of treating a disease associated with elevated blood uric acid levels comprising the administration of a therapeutically effective amount of 4-[2-(5-amino-1H-pyrazol-4-yl)-4-chlorophenoxy]-5-chloro-2-fluoro-N-(1,3-thiazol-4-yl)benzenesulfonamide or a pharmaceutically acceptable salt thereof to a subject in need of such treatment.

26. The method according to claim 25, wherein the subject is co-administered at least one additional pharmaceutically active agent selected from the group consisting of an anti-inflammatory agent, a xanthine oxidase inhibitor, a uricase and a uricosuric.

27. The method according to claim 26, wherein the subject is co-administered an anti-inflammatory agent selected from celecoxib, colchicine or a steroid.

28. The method according to claim 27, wherein the subject is co-administered colchicine.

29. The method according to claim 26, wherein the subject is co-administered a xanthine oxidase inhibitor selected from allopurinol, febuxostat, tisopurine, or ulodesine.

30. The method according to claim 26, wherein the subject is co-administered a uricase selected from pegloticase or rasburicase.

31. The method according to claim 26, wherein the subject is co-administered a uricosuric selected from benziodarone, isobromindione, probenecid, sulphinpyrazone, or benzbromarone.

32. The method for treating a disease associated with elevated blood uric acid levels according to claim 25, wherein the disease is hyperuricemia.

33. The method for treating a disease associated with elevated blood uric acid levels according to claim 25, wherein the disease is gout.

34. Crystalline 4-[2-(5-Amino-1H-pyrazol-4-yl)-4-chlorophenoxy]-5-chloro-2-fluoro-N-(1,3-thiazol-4-yl)benzenesulfonamide tosylate, exhibiting a Powder X-Ray Diffraction pattern (PXRD) having at least three characteristic two-theta (2θ) peaks selected from the group consisting of 9.0, 9.3, 10.0, 10.7, 11.6, 12.5, 12.9, 13.2, 13.8, 14.4, 16.0, 16.6, 17.5, 17.8, 18.1, 21.4 and 23.4° (+/−0.2° 2θ) by using CuKalpha1 X-ray radiation (wavelength=1.5406 Å).

35. The crystalline form according to claim 34, having at least four characteristic two-theta (2θ) peaks.

36. The crystalline form according to claim 34, having at least five characteristic two-theta (2θ) peaks.

37. The crystalline form according to claim 34, having at least six characteristic two-theta (2θ) peaks.

38. A pharmaceutical composition comprising the crystalline form according to claim 34, and a pharmaceutically acceptable carrier.

39. A method of treating pain comprising the administration of the crystalline form according to claim 34 to a subject in need of such treatment.

40. The method according to claim 39, wherein the pain is selected from neuropathic, nociceptive or inflammatory pain.

41. A method of treating hyperuricemia comprising the administration of the crystalline form according to claim 34 to a subject in need of such treatment.

42. A method of treating gout comprising the administration of the crystalline form according to claim 34 to a subject in need of such treatment.
